SEQUENCE LISTING (16 sequences)

SEQ ID NO: 1 - DNA, 1557 bp, Escherichia coli
atgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 2 - DNA, 43 bp, Artificial
  primer
gcagagctctaaggaggttacaatgatcccggacgtatcacag

SEQ ID NO: 3 - DNA, 37 bp, Artificial
  primer
gcgggtaccttatcaggcgtgtttttccagccacacc

SEQ ID NO: 4 - DNA, 951 bp, Escherichia coli
atgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 5 - DNA, 36 bp, Artificial
  primer
agcatcgtacatatgatcaagctcggcatcgtgatg

SEQ ID NO: 6 - DNA, 36 bp, Artificial
  primer
gcagaattctcattactgctgctgtaaacgtgcttc

SEQ ID NO: 7 - DNA, 2265 bp, Streptococcus agalactiae
catatgattattgaccgtctgctgcaacgctcccatagccatctgccgatcctgcaagccacctttggtctggaacgtgaatccctgcgcattcatcagccgacccagcgtgtggcccagacgccgcatccgaaaaccctgggctctcgcaactatcacccgtacatccaaacggattatagtgaaccgcagctggaactgattaccccgatcgccaaagactctcaggaagcaatccgttttctgaaagccatttcagatgttgcaggtcgctcgattaatcatgacgaatatctgtggccgctgagtatgccgccaaaagtccgtgaagaagatattcaaatcgctcagctggaagatgcgttcgaatatgactaccgcaaatatctggaaaaaacctacggcaaactgattcagtccatctcaggtattcactataacctgggcctgggtcaagaactgctgacctcgctgtttgaactgagccaggcggataacgccattgacttccagaatcaactgtatatgaaactgtctcagaattttctgcgttaccgctggctgctgacctatctgtacggcgccagtccggtggcagaagaagatttcctggaccagaaactgaacaatccggtccgttccctgcgcaactcacatctgggttatgtgaatcacaaagatattcgtatctcgtataccagcctgaaagattacgtgaacgacctggaaaatgctgttaaatctggccagctgattgcggaaaaagaattttatagcccggttcgtctgcgcggctctaaagcctgccgtaactatctggaaaaaggtatcacgtacctggaatttcgcaccttcgatctgaatccgtttagtccgattggtatcacccaggaaacggtggataccgttcacctgtttctgctggcgctgctgtggattgacagctctagtcacatcgatcaggacattaaagaagccaaccgcctgaatgatctgatcgcactgagccatccgctggaaaaactgccgaaccaggctccggtttcagatctggtcgacgcaatgcaatcggtgattcagcactttaatctgagcccgtattaccaagatctgctggaaagtgttaaacgtcagatccaatccccggaactgaccgttgcgggtcagctgctggaaatgattgaaggtctgtccctggaaaccttcggccagcgccagggtcagatctatcatgattacgcatgggaagctccgtatgcgctgaaaggctacgaaacgatggaactgagcacccaactgctgctgttcgatgttattcagaaaggtgtgaactttgaagttctggatgaacaagaccagttcctgaaactgtggcataattcccacatcgaatatgtgaaaaacggcaatatgacgtcaaaagataactacatcgttccgctggcgatggccaataaagtggttaccaagaaaattctggacgaaaaacactttccgacgccgttcggtgatgaatttaccgaccgtaaagaagcgctgaactatttttcgcaaatccaggataaaccgattgtcgtgaaaccgaaaagcacgaacttcggcctgggtatttctatctttaaaaccagtgccaatctggcatcctacgaaaaagctattgatatcgcgtttacggaagacagcgcgatcctggtcgaagaatatattgaaggcaccgaataccgtttctttgtgctggagggtgattgtatcgcggtcctgctgcgtgtggccgcaaatgttgttggtgacggtatccataccatttcccaactggtgaaactgaaaaaccagaatccgctgcgtggctatgatcaccgctcaccgctggaagttatcgaactgggtgaagtcgaacagctgatgctggaacagcaaggctacacggtgaactcgatcccgccggaaggtaccaaaattgaactgcgtcgcaactctaatatcagtacgggcggtgatagcattgacgttaccaatacgatggatccgacctataaacagctggcagctgaaatggcagaagctatgggcgcatgggtctgtggtgtggatctgattatcccgaacgctacgcaggcgtacagcaaagacaagaaaaacgcgacctgcattgaactgaactttaatccgctgatgtatatgcatacctactgtcaagaaggtccgggtcagagcatcacgccgcgcatcctggcaaaactgtttccggaactgtaatgagaattc

SEQ ID NO: 8 - DNA, 1440 bp, Acinetobacter johnsonii
catatggataccgaaacgattgcctccgccgtcctgaacgaagaacaactgtcgctggatctgattgaagcacaatacgctctgatgaacacccgcgatcagtcgaatgcgaaaagcctggtgattctggtttccggcatcgaactggccggcaaaggtgaagcagtcaaacagctgcgcgaatgggtggacccgcgttttctgtatgttaaagccgatccgccgcacctgtttaatctgaaacagccgttctggcaaccgtatacccgctttgttccggcggagggtcagattatggtctggttcggcaactggtacggtgatctgctggctaccgcgatgcacgcatcgaaaccgctggatgacacgctgtttgatgaatatgtcagcaatatgcgtgcgttcgaacaggatctgaaaaacaataacgtcgacgtgctgaaagtgtggtttgatctgtcctggaaatcactgcaaaaacgcctggatgacatggatccgtctgaagttcattggcacaaactgcatggcctggactggcgtaacaaaaaacagtacgataccctgcaaaaactgcgtacccgctttacggatgactggcagattatcgatggtgaagatgaagacctgcgcaatcacaacttcgcccaagcaattctgacggccctgcgtcattgcccggaacacgaaaagaaagcggccctgaaatggcagcaagcaccgattccggacatcctgacccagtttgaagtgccgcaagctgaagatgcgaactacaaatcagaactgaaaaaactgacgaaacaggttgctgatgcgatgcgttgtgatgaccgcaaagtggttatcgccttcgaaggcatggacgcagctggtaaaggcggtgcaattaaacgcatcgttaaaaaactggatccgcgtgaatacgaaattcataccatcgcggccccggaaaaatatgaactgcgtcgcccgtacctgtggcgcttttggtctaaactgcagagtgatgacattaccatcttcgatcgtacgtggtatggccgcgttctggtcgaacgtgtcgaaggttttgcgaccgaagtggaatggcagcgcgcctacgcagaaattaatcgttttgagaaaaacctgagctctagtcagacggtgctgattaaattctggctggctatcgataaagacgaacaagcagctcgttttaaagcccgcgaaagcaccccgcataaacgtttcaaaatcacggaagaagattggcgtaatcgcgacaaatgggatgactatctgaaagcggccgcagatatgttcgctcacaccgacacgtcctatgcgccgtggtacattatctcaaccaacgataaacagcaagctcgtatcgaagtcctgcgtgccatcctgaaacaactgaaagccgaccgtgacaccgactaatgagaattc

SEQ ID NO: 9 - protein, 518 aa, Escherichia coli
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 10 - protein, 316 aa, Escherichia coli [sequence omitted]

SEQ ID NO: 11 - DNA, 2253 bp, Streptococcus agalactiae
atgattatcgatcgactgttacaaagaagccactctcatctaccgattctacaagctacatttggcttagagagagaaagccttcgtattcaccaacccactcaaagggttgctcaaacaccccatcccaaaacattagggagtcgtaactatcatccttatatccaaacggattatagtgaacctcaattagaactcattacacctattgcaaaggatagccaagaagccattcgatttttgaaagctataagtgatgttgctggacgctctatcaaccacgatgaatacttatggccgctttctatgccacccaaagtgagggaagaagacatacagattgcccaattagaggatgcttttgaatatgattatcgcaaatatttggagaagacttatggaaaactaatacaatccatctctggaattcactataatctcggtttaggtcaagagttattaacttctctctttgaattgagtcaagcagataatgctattgattttcagaaccagctttacatgaaattgtctcaaaattttttacgttaccgttggttactaacttatctatacggagcgagcccagtagctgaagaagactttttagatcagaaactgaataaccctgtccggtcacttagaaatagtcacttaggttatgtgaatcataaagatattcgtatttcttatactagcttgaaggattatgtcaacgatctagaaaatgctgtaaaaagtgggcaattgattgctgagaaagaattttattcacctgttcgtctccgtggtagtaaggcctgtcgtaattatttagaaaaaggaattacatatttagagtttcgcacttttgatctcaacccatttagcccaatcggtataacgcaggaaactgttgatactgttcaccttttcttattggcgcttttgtggatagattctagtagtcatattgaccaagatatcaaggaagccaatagattaaatgaccttatagcacttagtcatccattagaaaaattacctaatcaagcaccagtttctgacttagtggatgctatgcaatctgttatccaacattttaacttatcaccttactatcaagacctattggaatctgtaaaaaggcaaattcagtcacctgaattaacggtagctggtcaacttttagaaatgattgaaggactttccttagaaacatttggacaaagacaaggacaaatttatcacgattatgcttgggaagctccctacgccttaaagggatatgaaacgatggaactttctacccaattgctactatttgacgttatccaaaaaggagtcaacttcgaagtgctggatgaacaagatcaattcctaaaattatggcacaatagtcatattgagtacgttaaaaatggtaatatgacctcaaaagataactatattgtaccgctcgctatggctaacaaagttgttacaaaaaaaatcctagatgaaaagcatttcccaactccttttggagatgaatttactgaccgtaaagaggcacttaactatttttcacaaatccaagataaaccaatcgtcgttaagccaaaatctacaaactttggtttaggcatttctatttttaaaacatcagctaacttagcttcttatgaaaaagcaattgacatcgcttttaccgaagatagtgctattcttgtggaagaatatattgagggtaccgaataccgtttcttcgtccttgagggggactgtattgctgtattgcttagggtagcagctaatgtcgttggagatggtattcatactattagtcagctagttaaacttaaaaaccaaaaccctctcagaggctatgatcatcgctctcctctagaggttattgagctcggagaagtagaacagttaatgttggaacaacaaggatacactgttaacagtatccctccagaaggaacaaaaatagaacttcgccgtaattctaatatttccactggcggtgactctatagatgtaacaaatacaatggatcctacatataaacaactagcagctgagatggcagaggctatgggggcttgggtttgtggagttgatttaattattcctaatgccactcaagcatactcaaaagataagaaaaatgctacttgcattgaactaaactttaacccattgatgtatatgcacacctactgtcaagaggggcctggtcaatctattacacctcgaattttggctaaactgttcccagaattataa

SEQ ID NO: 12 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 13 - DNA, 645 bp, Escherichia coli
atgcgtatcattctgcttggcgctccgggcgcggggaaagggactcaggctcagttcatcatggagaaatatggtattccgcaaatctccactggcgatatgctgcgtgctgcggtcaaatctggctccgagctgggtaaacaagcaaaagacattatggatgctggcaaactggtcaccgacgaactggtgatcgcgctggttaaagagcgcattgctcaggaagactgccgtaatggtttcctgttggacggcttcccgcgtaccattccgcaggcagacgcgatgaaagaagcgggcatcaatgttgattacgttctggaattcgacgtaccggacgaactgatcgttgaccgtatcgtcggtcgccgcgttcatgcgccgtctggtcgtgtttatcacgttaaattcaatccgccgaaagtagaaggcaaagacgacgttaccggtgaagaactgactacccgtaaagatgatcaggaagagaccgtacgtaaacgtctggttgaataccatcagatgacagcaccgctgatcggctactactccaaagaagcagaagcgggtaataccaaatacgcgaaagttgacggcaccaagccggttgctgaagttcgcgctgatctggaaaaaatcctcggctaa

SEQ ID NO: 14 - protein, 214 aa, Escherichia coli
MRIILLGAPGAGKGTQAQFIMEKYGIPQISTGDMLRAAVKSGSELGKQAKDIMDAGKLVTDELVIALVKERIAQEDCRNGFLLDGFPRTIPQADAMKEAGINVDYVLEFDVPDELIVDRIVGRRVHAPSGRVYHVKFNPPKVEGKDDVTGEELTTRKDDQEETVRKRLVEYHQMTAPLIGYYSKEAEAGNTKYAKVDGTKPVAEVRADLEKILG

SEQ ID NO: 15 - DNA, 1428 bp, Acinetobacter johnsonii
atggatacagaaacgatcgccagtgcagtgctgaatgaagaacagctttcactggacttaattgaagcgcaatatgcgttgatgaatacccgtgatcagagcaatgcaaaaagtttagtgattttggtcagtggaatcgaacttgcgggtaaaggcgaagcggtgaaacagctccgcgaatgggtcgatcctcgttttttatatgtcaaagccgatccaccgcatctgtttaatctaaaacagcctttttggcagccctatacccgatttgtgcctgccgaagggcaaattatggtgtggtttggtaattggtatggggatttgttggctacggccatgcatgcttcaaagcctttagatgacactttgtttgatgaatacgtcagcaatatgcgggcttttgaacaggacttaaaaaataacaacgtagatgtcttaaaagtttggttcgatttgtcgtggaagtctctgcaaaagcgtctagatgatatggacccgagcgaagtgcattggcataagttgcatgggctagactggcgcaataaaaaacaatatgacaccttacaaaagctacgtacgcgcttcaccgatgactggcaaatcattgatggtgaagatgaggatttgcgtaatcacaattttgcacaagcaattttaacggcactacgacactgcccagagcatgaaaaaaaggccgcgctaaaatggcagcaagcaccaataccagatattctgactcagtttgaagtccctcaagctgaggatgcgaactataaatcagaattgaaaaaactcaccaaacaagtggccgatgccatgcgctgtgatgaccgtaaagtggtgattgcttttgaaggtatggatgctgcgggtaaagggggggcgattaagcgtattgtgaaaaagctcgacccacgagaatatgaaattcataccattgccgcacctgaaaaatatgagttacgccgtccttatctgtggcgtttttggagcaaattgcagtcggatgacatcactatttttgatcggacgtggtatggacgcgttttagtcgagcgggtagaaggcttcgcaaccgaggtagagtggcaacgcgcttatgcggaaatcaatcgttttgaaaaaaacctcagtagcagccaaaccgtgctgattaagttttggctggcgattgataaagatgaacaagcagcgcgttttaaagcccgcgaaagtactccgcataaacgttttaaaattaccgaagaagattggcgcaatcgcgacaaatgggatgactatttaaaggcagccgcggatatgtttgcgcataccgacaccagctatgcgccttggtatattatttccaccaatgataaacaacaggcccgcattgaagtcttaagggcaattttaaaacagctcaaagcggatcgcgacacggattaa

SEQ ID NO: 16 - protein, 475 aa, Acinetobacter johnsonii
MDTETIASAVLNEEQLSLDLIEAQYALMNTRDQSNAKSLVILVSGIELAGKGEAVKQLREWVDPRFLYVKADPPHLFNLKQPFWQPYTRFVPAEGQIMVWFGNWYGDLLATAMHASKPLDDTLFDEYVSNMRAFEQDLKNNNVDVLKVWFDLSWKSLQKRLDDMDPSEVHWHKLHGLDWRNKKQYDTLQKLRTRFTDDWQIIDGEDEDLRNHNFAQAILTALRHCPEHEKKAALKWQQAPIPDILTQFEVPQAEDANYKSELKKLTKQVADAMRCDDRKVVIAFEGMDAAGKGGAIKRIVKKLDPREYEIHTIAAPEKYELRRPYLWRFWSKLQSDDITIFDRTWYGRVLVERVEGFATEVEWQRAYAEINRFEKNLSSSQTVLIKFWLAIDKDEQAARFKARESTPHKRFKITEEDWRNRDKWDDYLKAAADMFAHTDTSYAPWYIISTNDKQQARIEVLRAILKQLKADRDTD

(19) United States
(12) Patent Application Publication
(10) Pub. No.: US 2017/0145466 A1
(43) Pub. Date: May 25, 2017

(54) METHOD FOR PRODUCING OXIDIZED GAMMA-GLUTAMYLCYSTEINE AND OXIDIZED GLUTATHIONE

(71) Applicant: KANEKA CORPORATION, Osaka-shi, Osaka (JP)

(72) Inventors: Masutoshi NOJIRI, Takasago-shi (JP); Yoshihiko YASOHARA, Takasago-shi (JP)

(73) Assignee: KANEKA CORPORATION, Osaka-shi, Osaka (JP)

(21) Appl. No.: 15/323,144

(22) PCT Filed: Jul. 2, 2015
(86) PCT No.: PCT/JP2015/069141

§ 371 Date: Dec. 30, 2016

(30) Foreign Application Priority Data
Jul. 2, 2014 (JP) 2014-137202

(51) Int. Cl. C12P 21/02

(52) U.S. Cl. CPC C12P 21/02

(57) ABSTRACT

The technical problem to be solved by the present invention is to provide a method for producing oxidized glutathione, GSSG and a precursor thereof, i.e., oxidized γ-glutamylcysteine, by a simple process. As a means for solving the problem, the method for producing GSSG according to the present invention comprises step A′ of reacting L-cystine and L-glutamic acid to produce oxidized γ-glutamylcysteine and step B′ of reacting oxidized γ-glutamylcysteine and glycine to produce GSSG.

### TECHNICAL FIELD

The present invention relates to a method for producing oxidized γ-glutamylcysteine.

The present invention also relates to a method for producing oxidized glutathione.

### BACKGROUND ART

Glutathione is a peptide consisting of three amino acids, i.e., L-cysteine, L-glutamic acid and glycine and present in a wide variety of living organisms including not only humans but also other animals, plants and microorganisms, and is an important compound for living organisms involved in, e.g., removal of reactive oxygen species, detoxification and amino acid metabolism.

Glutathione is present in vivo in the form of either one of reduced glutathione (N—(N-γ-L-glutamyl-L-cysteinyl)glycine, in which the thiol group of the L-cysteine residue is reduced in the form of SH (hereinafter will be sometimes referred to as “GSH”) and oxidized glutathione in which the thiol group of the L-cysteine residue is oxidized to form a disulfide bond between two glutathione molecules (hereinafter will be sometimes referred to as “GSSG”).

It is known that GSSG has higher preservation stability than GSH (e.g., Patent Literature 1). Glutathione is often present in the form of GSH (reduced glutathione) in vivo in most cases. GSH is easily produced on a large scale by using e.g., a yeast; however, GSSG has been not yet easily produced on a large scale with high efficiency. In conventional (general) methods, GSH is produced first and then GSH is chemically or biologically oxidized to produce GSSG. For example, in the method disclosed in Patent Literature 1, a yeast extract containing GSH is first produced; and then, while controlling the pH value of the yeast extract to be 6 or more to less than 11, GSH is converted into GSSG in the presence of dissolved oxygen.

### CITATION LIST

### Patent Literatures

### Patent Literature 1: JP Patent No. 3160335

### Patent Literature 2: JP Patent Publication (Kohyo) No. 2013-505712

### Patent Literature 3: JP Patent Publication (Kokai) No. 2013-188177

### SUMMARY OF INVENTION

### Technical Problem

The technical problem to be solved by the present invention is to provide a method for producing GSSG and a precursor compound thereof by a simple process.

### Solution to Problem

The present inventors found that GSSG and a precursor thereof, i.e., oxidized γ-glutamylcysteine, can be produced from relatively inexpensive starting material, L-cystine, by a simple process and reached completion of the present invention. L-cystine is a compound consisting of two L-cysteine molecules bound via a disulfide bond by oxidation of the thiol groups of them. It is known that L-cystine is used, for example, as a starting material for producing L-cysteine (Patent Literature 2) and as a starting material for producing GSH through lactic fermentation (Patent Literature 3); however, a method of producing GSSG directly from L-cystine has not yet been present.

More specifically, the present invention includes the following invention.

(1) A method for producing oxidized γ-glutamylcysteine, characterized by comprising step A of reacting L-cystine and L-glutamic acid in the presence of at least one enzyme selected from the group consisting of γ-glutamylcysteine synthetase and a bifunctional glutathione synthetase, and adenosine triphosphate (ATP, also referred to as adenosine 5′-triphosphate) to produce oxidized γ-glutamylcysteine.

According to the present invention, oxidized γ-glutamylcysteine can be directly produced from relatively inexpensive L-cystine.

(2) The method according to (1), in which the step A is carried out in conjugation with an ATP regeneration reaction for regenerating adenosine diphosphate (ADP, also referred to as adenosine 5′-diphosphate) into ATP.

In the step A, ATP is consumed to produce ADP; however, the step A according to the present invention can be continuously performed while regenerating ATP.

(3) The method according to (1) or (2), in which the γ-glutamylcysteine synthetase is derived from Escherichia coli.

Such γ-glutamylcysteine synthetase is preferable since it has a particularly high activity to produce oxidized γ-glutamylcysteine from L-cystine and L-glutamic acid.

(4) The method according to (1) or (2), in which the bifunctional glutathione synthetase is derived from Streptococcus agalactiae.

Such a bifunctional glutathione synthetase is preferable since it has a particularly high activity to produce oxidized γ-glutamylcysteine from L-cystine and L-glutamic acid.

(5) A method for producing oxidized glutathione, characterized by comprising step B of reacting oxidized γ-glutamylcysteine and glycine in the presence of a glutathione synthetase and adenosine triphosphate (ATP) to produce oxidized glutathione.

According to the present invention, oxidized glutathione can be produced by a simple process.

(6) The method according to (5), in which the step B is carried out in conjugation with an ATP regeneration reaction for regenerating adenosine diphosphate (ADP) into ATP.

In the step B, ATP is consumed to produce ADP; however, the step B according to the present invention can be continuously performed while regenerating ATP.

(7) The method according to (5) or (6), in which the glutathione synthetase is derived from Escherichia coli.

Such a glutathione synthetase is preferable since it has a particularly high activity to produce oxidized glutathione from oxidized γ-glutamylcysteine and glycine.

(8) The method according to any one of (5) to (7), further having step A of reacting L-cystine and L-glutamic acid in the presence of at least one enzyme selected from the group consisting of γ-glutamylcysteine synthetase and a bifunctional glutathione synthetase, and adenosine triphosphate (ATP) to produce oxidized γ-glutamylcysteine, in which oxidized γ-glutamylcysteine used in the step B is produced by the step A.

According to the present invention, oxidized glutathione can be produced from relatively inexpensive L-cystine by a simple process.

(9) The method according to (8), in which the step A is carried out in conjugation with an ATP regeneration reaction for regenerating adenosine diphosphate (ADP) into ATP.

In the step A, ATP is consumed to produce ADP; however, the step A according to the present invention can be continuously carried out while regenerating ATP.

(10) The method according to (8) or (9), in which the γ-glutamylcysteine synthetase is derived from Escherichia coli.

Such γ-glutamylcysteine synthetase is preferable since it has a particularly high activity to produce oxidized γ-glutamylcysteine from L-cystine and L-glutamic acid.

(11) The method according to (8) or (9), in which the bifunctional glutathione synthetase is derived from Streptococcus agalactiae.

Such a bifunctional glutathione synthetase is preferable since it has a particularly high activity to produce oxidized γ-glutamylcysteine from L-cystine and L-glutamic acid.

(12) A method for producing oxidized γ-glutamylcysteine characterized by comprising step A′ of reacting L-cystine and L-glutamic acid to produce oxidized γ-glutamylcysteine.

According to the present invention, oxidized γ-glutamylcysteine can be directly produced from relatively inexpensive L-cystine. In the present invention, it is more preferable that the step A′ is the step A according to (1).

(13) A method for producing oxidized glutathione, characterized by comprising step B′ of reacting oxidized γ-glutamylcysteine and glycine to produce oxidized glutathione.

According to the present invention, oxidized glutathione can be produced by a simple process. In the present invention, it is more preferable that the step B′ is the step B according to (5).

(14) The method according to (13), further having step A′ of reacting L-cystine and L-glutamic acid to produce oxidized γ-glutamylcysteine, in which oxidized γ-glutamylcysteine used in the step B′ is produced by the step A′.

According to the present invention, oxidized glutathione can be produced from relatively inexpensive L-cystine by a simple process. In the present invention, it is more preferable that the step A′ is the step A according to (1).

In the specification, the compounds represented by terms: e.g., “L-cystine”, “L-glutamic acid”, “glycine” “oxidized γ-glutamylcysteine”, “oxidized glutathione”, “L-cysteine”, “glutamylcysteine”, “glutathione”, “adenosine triphosphate”, “adenosine diphosphate”, “adenosine monophosphate”, “polyphosphoric acid” and “condensed phosphoric acid” may take any forms including free form, a salt (e g, a sodium salt and a potassium salt), a solvate (e.g., a hydrate), and an ion.

Oxidized γ-glutamylcysteine in its free form is represented by the following formula:

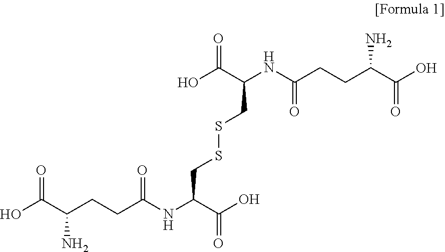 [SMILES: N[C@@H](CCC(=O)N[C@@H](CSSC[C@H](NC(=O)CC[C@H](N)C(=O)O)C(=O)O)C(=O)O)C(=O)O]

Oxidized glutathione in its free form is represented by the following formula:

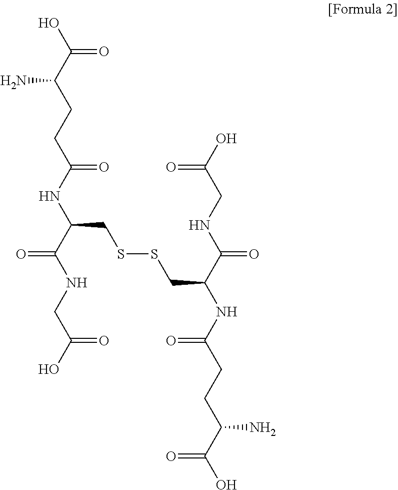 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

The teachings of Japanese Patent Application No. 2014-137202, on which the priority of the present application is based, are incorporated into this specification.

### Advantageous Effects of Invention

According to the present invention, it is possible to produce oxidized glutathione (GSSG) and oxidized γ-glutamylcysteine from an inexpensive material by a simple process.

### DESCRIPTION OF EMBODIMENTS

### <Enzymes Used in the Present Invention>

As used herein, γ-glutamylcysteine synthetase will be sometimes referred to simply as “GSH I”, glutathione synthetase as “GSH II”, a bifunctional glutathione synthetase as “GSH F”, adenylate kinase as “ADK”, and polyphosphate-dependent AMP transferase as “PAP”.

### <GSH I>

The γ-glutamylcysteine synthetase (GSH I) used in the present invention is an enzyme having an activity to recognize L-Cys as a substrate and to catalyze a reaction for producing γ-Glu-Cys by mediating binding the L-Cys to L-Glu in the presence of ATP. GSH I may be any enzyme having this activity. The source, structure and other characteristics of GSH I are not limited. In the present invention, this activity is referred to as γ-glutamylcysteine synthetase activity. One unit (1 U) of the activity means the activity to produce 1 μmol of γ-glutamylcysteine at 30° C. for one minute and is measured in the following measurement condition. The present invention is based on a new finding that γ-glutamylcysteine synthetase also has an activity to produce oxidized γ-glutamylcysteine by reacting L-cystine and L-glutamic acid in the presence of ATP.

### (Measurement Condition)

The reaction comprises adding an enzyme solution to a 50 mM tris hydrochloride buffer solution (pH8.0) containing 10 mM ATP, 15 mM L-glutamic acid, 15 mM L-cysteine and 10 mM magnesium sulfate and then keeping the reaction solution at 30° C. The reaction is terminated by adding 6N hydrochloric acid. The γ-glutamylcysteine in the reaction solution is quantified by high-performance liquid chromatography.

The conditions for the high-performance liquid chromatography are as follows. In the conditions, reduced glutathione (GSH), reduced γ-glutamylcysteine (γ-GC), oxidized γ-GC and oxidized glutathione (GSSG) sequentially elute in this order.

### [HPLC Conditions]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptanesulfonate (3.6 g) with distilled water (1.8 L), controlling the pH of the solution with phosphoric acid to be pH2.8, adding methanol (186 ml) and dissolving it;

Flow rate: 1.0 ml/minute;

Column temperature: 40° C.;

Measurement wavelength: 210 nm.

GSH I having a γ-glutamylcysteine synthetase activity (specific activity) of 0.5 U or more per 1 mg of protein is preferably used as GSH I.

The source from which GSH I is derived is not limited and GSH I derived from e.g., microorganisms, animals and plants can be used. GSH I derived from a microorganism is preferable, particularly GSH I derived from an intestinal microbe, such as Escherichia coli, a corynebacterium and an eukaryotic microorganism, such as a yeast, are preferable.

The nucleotide sequence of GSH I derived from Escherichia coli and the amino acid sequence encoded by the nucleotide sequence are specifically represented by SEQ ID NO: 1 and SEQ ID NO: 9, respectively.

GSH I that can be used includes not only GSH I consisting of the amino acid sequence represented by SEQ ID NO: 9 but also other polypeptides having GSH I activity, such as an active mutant thereof and orthologue from other species. Said other polypeptides having GSH I activity include a polypeptide exhibiting an activity of preferably 10% or more, preferably 40% or more, more preferably 60% or more, more preferably 80% or more and further preferably 90% or more relative to the activity of the GSH I consisting of the amino acid sequence represented by SEQ ID NO: 9 in the aforementioned activity measurement conditions; and preferably, a polypeptide exhibiting an activity of preferably 10% or more, preferably 40% or more, more preferably 60% or more, more preferably 80% or more and further preferably 90% or more relative to the activity to produce oxidized γ-glutamylcysteine from L-cystine and L-glutamic acid in the presence of ATP of the GSH I consisting of the amino acid sequence represented by SEQ ID NO: 9. Examples of said other polypeptides having GSH I activity, such as an active mutant thereof and orthologue from other species, include a polypeptide consisting of an amino acid sequence having addition, deletion or substitution of one or several amino acids in the amino acid sequence represented by SEQ ID NO: 9 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 9); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 9. Further examples of said other polypeptides that can be used herein include a GSH I-active fragment of at least one polypeptide selected from the group consisting of the polypeptide consisting of the amino acid sequence represented by SEQ ID NO: 9; a polypeptide consisting of an amino acid sequence having addition, deletion or substitution of one or several amino acids in the amino acid sequence represented by SEQ ID NO: 9; and a polypeptide consisting of the amino acid sequence having the aforementioned amino acid identity with the amino acid sequence represented by SEQ ID NO: 9. The fragment may be a polypeptide consisting of amino acids of preferably 250 or more, more preferably 300 or more, more preferably 400 or more and more preferably 500 or more. The fragment also has an activity to produce oxidized γ-glutamylcysteine from L-cystine and L-glutamic acid in the presence of ATP. In the specification, “several” refers to, for example, 2 to 20, 2 to 15, 2 to 10, 2 to 7, 2 to 5, 2 to 4 or 2 to 3. The “amino acid identity” refers to the percentage (%) of the number of identical amino acid residues relative to the total number of amino acid residues of the protein represented by SEQ ID NO: 9, when two amino acid sequences are aligned so as to obtain a highest degree of matching of amino acids between the two sequences, if necessary by introducing a gap. The amino acid identity can be calculated by using a protein search system, such as BLAST and FASTA (Karlin, S. et al., 1993, Proc. Natl. Acad. Sci. USA, 90: 5873-5877; Altschul, S. F. et al., 1990, J. Mol. Biol., 215: 403-410; Pearson, W. R. et al., 1988, Proc. Natl. Acad. Sci. USA, 85: 2444-2448). Furthermore, the substitution of amino acids is made preferably in accordance with conservative amino acid substitution. The “conservative amino acid substitution” refers to substitution between amino acids having analogous properties with respect to e.g., charge, side chain, polarity and aromaticity. The amino acids analogous in property are categorized as follows: a basic amino acid (arginine, lysine, histidine); an acidic amino acid (aspartic acid, glutamic acid); an uncharged polar amino acid (glycine, asparagine, glutamine, serine, threonine, cysteine, tyrosine); a nonpolar amino acid (leucine, isoleucine, alanine, valine, proline, phenylalanine, tryptophan, methionine); a branched-chain amino acid (leucine, valine, isoleucine); and an aromatic amino acid (phenylalanine, tyrosine, tryptophan, histidine). The aforementioned polypeptides each may be chemically modified.

The nucleotide sequence of a gene (DNA or RNA) encoding GSH I that can be used for preparation of GSH I is not limited to the nucleotide sequence represented by SEQ ID NO: 1. The nucleotide sequence may be any nucleotide sequence encoding the amino acid sequence of a desired GSH I and being suited to the host organism.

### <GSH II>

The glutathione synthetase (GSH IT) used in the present invention is an enzyme having an activity to recognize γ-Glu-Cys as a substrate and to catalyze a reaction for producing γ-Glu-Cys-Gly by mediating binding the γ-Glu-Cys to Gly in the presence of ATP. GSH TI may be any enzyme having this activity. The source, structure and other characteristics of GSH II are not limited. In the present invention, the activity is referred to as glutathione synthetase activity. One unit (1 U) of the activity means the activity to produce 1 μmol of glutathione at 30° C. for one minute and is measured in the following measurement conditions. The present invention is based on a new finding that glutathione synthetase also has an activity to produce oxidized glutathione by reacting oxidized γ-glutamylcysteine and glycine in the presence of ATP.

### (Measurement Condition)

The reaction comprises adding an enzyme solution to a 50 mM tris hydrochloride buffer solution (pH8.0) containing 10 mM ATP, 15 mM γ-glutamylcysteine, 15 mM glycine and 10 mM magnesium sulfate and then keeping the reaction solution at 30° C. The reaction is terminated by adding 6N hydrochloric acid. The glutathione in the reaction solution is quantified by high-performance liquid chromatography.

The same conditions for the high-performance liquid chromatography as mentioned in the above GSH I activity measurement method are used.

GSH II having a glutathione synthetase activity (specific activity) of 0.5 U or more per 1 mg of protein is preferably used as GSH II.

The source from which GSH II is derived is not limited and GSH II derived from e.g., microorganisms, animals and plants can be used. GSH II derived from a microorganism is preferable, particularly GSH II derived from an intestinal microbe, such as Escherichia coli, a corynebacterium and an eukaryotic microorganism, such as yeasts, are preferable.

The nucleotide sequence of GSH II derived from Escherichia coli and the amino acid sequence encoded by the nucleotide sequence are specifically represented by SEQ ID NO: 4 and SEQ ID NO: 10, respectively.

GSH II that can be used includes not only GSH II consisting of the amino acid sequence represented by SEQ ID NO: 10 but also other polypeptides having GSH II activity, such as an active mutant thereof and orthologue from other species. Said other polypeptides having GSH II activity include a polypeptide exhibiting an activity of preferably 10% or more, preferably 40% or more, more preferably 60% or more, more preferably 80% or more and further preferably 90% or more relative to the activity of the GSH II consisting of the amino acid sequence represented by SEQ ID NO: 10 in the aforementioned activity measurement conditions; and preferably, a polypeptide exhibiting an activity of preferably 10% or more, preferably 40% or more, more preferably 60% or more, more preferably 80% or more, and further preferably 90% or more relative to the activity to produce oxidized glutathione from oxidized γ-glutamylcysteine and glycine in the presence of ATP of the GSH II consisting of the amino acid sequence represented by SEQ ID NO: 10. Examples of said other polypeptides having GSH II activity, such as an active mutant thereof and orthologue from other species, include a polypeptide consisting of an amino acid sequence having addition, deletion or substitution of one or several amino acids in the amino acid sequence represented by SEQ ID NO: 10 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 10); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO:10. Further examples of said other polypeptides that can be used herein include a GSH II-active fragment of at least one polypeptide selected from the group consisting of the polypeptide consisting of the amino acid sequence represented by SEQ ID NO: 10; a polypeptide consisting of an amino acid sequence having addition, deletion or substitution of one or several amino acids in the amino acid sequence represented by SEQ ID NO: 10; and a polypeptide consisting of the amino acid sequence having the aforementioned amino acid identity with the amino acid sequence represented by SEQ ID NO: 10. The fragment may be a polypeptide consisting of amino acids of preferably 150 or more, more preferably 200 or more, more preferably 300 or more. The fragment also has an activity to produce oxidized glutathione from oxidized γ-glutamylcysteine and glycine in the presence of ATP. The “amino acid identity” refers to the percentage (%) of the number of identical amino acid residues relative to the total number of amino acid residues of the protein represented by SEQ ID NO: 10, when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the GSH II that can be used for preparation of GSH II is not limited to the nucleotide sequence represented by SEQ ID NO: 4. The nucleotide sequence may be any nucleotide sequence encoding the amino acid sequence of a desired GSH II and being suited to the host organism.

### <GSH F>

The bifunctional glutathione synthetase (GSH F) used in the present invention is an enzyme having two activities in combination, one of which is an activity to recognize L-Cys as a substrate and to catalyze a reaction for producing γ-Glu-Cys by mediating binding the L-Cys to L-Glu in the presence of ATP, and the other of which is an activity to recognize γ-Glu-Cys as a substrate and to catalyze a reaction for producing γ-Glu-Cys-Gly by mediating binding the γ-Glu-Cys to Gly in the presence of ATP. GSH F may be any enzyme having these two activities in combination. The source, structure and other characteristics of GSH F are not limited. In the present invention, the combination of the activities is referred to as bifunctional glutathione synthetase activity. One unit (1 U) of the activity means the activity to produce 1 μmol of γ-Glu-Cys-Gly (glutathione) at 30° C. for one minute and is measured in the following measurement conditions. The present invention is based on a new finding that bifunctional glutathione synthetase also has an activity to produce oxidized γ-glutamylcysteine by reacting L-cystine and L-glutamic acid in the presence of ATP.

### (Measurement Condition)

The reaction comprises adding an enzyme solution to a 50 mM tris hydrochloride buffer solution (pH8.0) containing 10 mM ATP, 15 mM L-glutamic acid, 15 mM L-cysteine, 15 mM glycine and 10 mM magnesium sulfate and then keeping the reaction solution at 30° C. The reaction is terminated by adding 6N hydrochloric acid. The glutathione in the reaction solution is quantified by high-performance liquid chromatography.

The same conditions for the high-performance liquid chromatography as mentioned in GSH I activity measurement method are used.

GSH F having a glutathione synthetase activity (specific activity) of 0.5 U or more per 1 mg of protein is preferably used as GSH F.

The source from which GSH F is derived is not limited and GSH F derived from e.g., microorganisms, animals and plants can be used. GSH F derived from a microorganism is preferable, particularly GSH F derived from a bacterium, and more specifically GSH F derived from at least one selected from the group consisting of Streptococcus bacteria, such as Streptococcus agalactiae, Streptococcus mutans, Streptococcus suis and Streptococcus thermophilus; Lactobacillus bacteria, such as Lactobacillus plantarum; Desulfotalea bacteria, such as Desulfotalea psychrophile, Clostridium bacteria, such as Clostridium perfringens; Listeria bacteria, such as Listeria innocua and Listeria monocytogenes; Enterococcus bacteria, such as Enterococcus faecalis and Enterococcus faecium; Pasteurella bacteria, such as Pasteurella multocida; Mannheimia bacteria, such as Mannheimia succiniciprodecens; and Haemophilus bacteria, such as Haemophilus somnus, is preferable.

The nucleotide sequence of GSH F derived from Streptococcus agalactiae and the amino acid sequence encoded by the nucleotide sequence are specifically represented by SEQ ID NO: 11 and SEQ ID NO: 12, respectively. Furthermore, the nucleotide sequence consisting of 4th base to 2253rd base of SEQ ID NO: 7 is an example of a nucleotide sequence encoding GSH F derived from Streptococcus agalactiae consisting of amino acid sequence represented by SEQ ID NO: 12 and being suited in accordance with the use frequency of codons in Escherichia coli.

GSH F that can be used includes not only GSH F consisting of the amino acid sequence represented by SEQ ID NO: 12 but also other polypeptides having GSH F activity, such as an active mutant thereof and orthologue from other species. Said other polypeptides having GSH F activity includes a polypeptide exhibiting an activity of preferably 10% or more, preferably 40% or more, more preferably 60% or more, more preferably 80% or more and further preferably 90% or more relative to the activity of the GSH F consisting of the amino acid sequence represented by SEQ ID NO: 12 in the aforementioned activity measurement conditions; and, preferably, a polypeptide exhibiting an activity of preferably 10% or more, preferably 40% or more, more preferably 60% or more, more preferably 80% or more, and further preferably 90% or more relative to the activity to produce oxidized γ-glutamylcysteine from L-cystine and L-glutamic acid in the presence of ATP of the GSH F consisting of the amino acid sequence represented by SEQ ID NO: 12. Examples of said other polypeptides having GSH F activity, such as an active mutant thereof and orthologue from other species, include a polypeptide consisting of an amino acid sequence having addition, deletion or substitution of one or several amino acids in the amino acid sequence represented by SEQ ID NO: 12 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO:12); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 12. Further examples of said other polypeptides that can be used herein include a GSH F-active fragment of at least one polypeptide selected from the group consisting of the polypeptide consisting of the amino acid sequence represented by SEQ ID NO: 12; a polypeptide consisting of an amino acid sequence having addition, deletion or substitution of one or several amino acids in the amino acid sequence represented by SEQ ID NO: 12; and a polypeptide consisting of the amino acid sequence having the aforementioned amino acid identity with the amino acid sequence represented by SEQ ID NO: 12. The fragment may be a polypeptide consisting of amino acids of preferably 400 or more, more preferably 500 or more, and more preferably 600 or more, more preferably 700 or more and more preferably 730 or more. The fragment also has an activity to produce oxidized γ-glutamylcysteine from L-cystine and L-glutamic acid in the presence of ATP. The “amino acid identity” refers to the percentage (%) of the number of identical amino acid residues relative to the total number of amino acid residues of the protein represented by SEQ ID NO: 12 when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the GSH F that can be used for preparation of GSH F is not limited to the nucleotide sequence represented by SEQ ID NO: 11. The nucleotide sequence may be any nucleotide sequence encoding the amino acid sequence of a desired GSH F and being suited to the host organism.

### <ADK>

Adenylate kinase (ADK) used in the present invention is an enzyme having an activity to catalyze a reaction for producing one ATP molecule and one AMP molecule from two ADP molecules. ADK may be any enzyme having this activity. The source, structure and other characteristics of ADK are not limited. In the present invention, the activity is referred to as ADK activity. One unit (1 U) of the activity means the activity to produce 1 μmol of AMP at 30° C. for one minute and is measured in the following measurement conditions.

### (Measurement Condition)

The reaction comprises adding an enzyme solution to a 50 mM tris hydrochloride buffer solution (pH8.0) containing 10 mM ADP and 70 mM magnesium sulfate, and then keeping the reaction solution at 30° C. The reaction is terminated by adding 6N hydrochloric acid. The AMP in the reaction solution is quantified by high-performance liquid chromatography.

The conditions of the high-performance liquid chromatography are as follows. In the conditions, adenosine triphosphate (ATP), adenosine diphosphate (ADP) and adenosine monophosphate (5′-adenyl acid) (AMP) sequentially elute in this order.

### [HPLC Conditions]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptanesulfonate (3.6 g) with distilled water (1.8 L), controlling the pH of the solution with phosphoric acid to be pH2.8, adding methanol (186 ml) and dissolving it;

Flow rate: 1.0 ml/minute;

Column temperature: 40° C.;

Measurement wavelength: 210 nm.

ADK having an ADK activity (specific activity) of 20 U or more per 1 mg of protein is preferably used as ADK.

The source from which ADK is derived is not limited and ADK derived from e.g., microorganisms, animals and plants can be used. ADK derived from a microorganism is preferable, and particularly ADK derived from a bacterium, and more specifically ADK derived from Escherichia coli is preferable.

The nucleotide sequence of ADK derived from Escherichia coli and the amino acid sequence encoded by the nucleotide sequence are specifically represented by SEQ ID NO: 13 and SEQ ID NO: 14, respectively.

ADK that can be used includes not only ADK consisting of the amino acid sequence represented by SEQ ID NO: 14 but also other polypeptides having ADK activity, such as an active mutant thereof and orthologue from other species. Said other polypeptides having ADK activity are preferably a polypeptide exhibiting an activity of preferably 10% or more, preferably 40% or more, more preferably 60% or more, more preferably 80% or more and further preferably 90% or more relative to the activity of the ADK consisting of the amino acid sequence represented by SEQ ID NO: 14 in the aforementioned activity measurement conditions. Examples of said other polypeptides having ADK activity, such as an active mutant thereof and orthologue from other species, include a polypeptide consisting of an amino acid sequence having addition, deletion or substitution of one or several amino acids in the amino acid sequence represented by SEQ ID NO: 14 (a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 14) and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 14. Further examples of said other polypeptides that can be used herein include a ADK-active fragment of at least one polypeptide selected from the group consisting of the polypeptide consisting of the amino acid sequence represented by SEQ ID NO: 14; a polypeptide consisting of an amino acid sequence having addition, deletion or substitution of one or several amino acids in the amino acid sequence represented by SEQ ID NO: 14; and a polypeptide consisting of the amino acid sequence having the aforementioned amino acid identity with the amino acid sequence represented by SEQ ID NO: 14. The fragment may be a polypeptide consisting of amino acids of preferably 100 or more, more preferably 150 or more, more preferably 200 or more. The “amino acid identity” refers to the percentage (%) of the number of identical amino acid residues relative to the total number of amino acid residues of the protein represented by SEQ ID NO: 14, when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be chemically modified.

The nucleotide sequence of a gene (DNA or RNA) encoding the ADK that can be used for preparation of ADK is not limited to the nucleotide sequence represented by SEQ ID NO: 13. The nucleotide sequence may be any nucleotide sequence encoding the amino acid sequence of a desired ADK and being suited to the host organism.

### <PAP>

Polyphosphate-dependent AMP transferase (PAP) used in the present invention is an enzyme having an activity to catalyze a reaction for phosphorylating AMP using a polyphosphoric acid as a phosphoric acid donor to produce ADP. PAP may be any enzyme having this activity. The source, structure and other characteristics of PAP are not limited. In the present invention, the activity is referred to as PAP activity. One unit (1 U) of the activity means the activity to produce 1 μmol of ADP at 30° C. for one minute and is measured in the following measurement condition.

### (Measurement Condition)

The reaction comprises adding an enzyme solution to a 50 mM tris hydrochloride buffer solution (pH8.0) containing 5 mM sodium metaphosphate, 10 mM AMP and 70 mM magnesium sulfate and then keeping the reaction solution at 30° C. The reaction is terminated by adding 6N hydrochloric acid. ADP in the reaction solution is quantified by high-performance liquid chromatography.

The same conditions for the high-performance liquid chromatography as mentioned in the above activity measurement method for ADK are used.

PAP having a PAP activity (specific activity) of 20 U or more per 1 mg of protein is preferably used as PAP.

The source of PAP is not limited and PAP derived from e.g., microorganisms, animals and plants can be used. PAP derived from a microorganism is preferable, and particularly PAP derived from a bacterium, and more specifically PAP derived from Acinetobacter johnsonii is preferable.

The nucleotide sequence of PAP derived from Acinetobacter johnsonii and the amino acid sequence encoded by the nucleotide sequence are specifically represented by SEQ ID NO: 15 and SEQ ID NO: 16, respectively. Furthermore, the nucleotide sequence consisting of 4th base to 1428th base of SEQ ID NO: 8 is an example of a nucleotide sequence encoding PAP derived from Acinetobacter johnsonii consisting of amino acid sequence represented by SEQ ID NO: 16 and, and being suited in accordance with the use frequency of codons in Escherichia coli.

PAP that can be used includes not only PAP consisting of the amino acid sequence represented by SEQ ID NO: 16 but also other polypeptides having PAP activity, such as an active mutant thereof and orthologue from other species. Said other polypeptides having PAP activity include a polypeptide exhibiting an activity of preferably 10% or more, preferably 40% or more, more preferably 60% or more, more preferably 80% or more and further preferably 90% or more relative to the activity of the PAP consisting of the amino acid sequence represented by SEQ ID NO: 16 in the aforementioned activity measurement conditions. Examples of said other polypeptides having PAP activity, such as an active mutant thereof and orthologue from other species, include a polypeptide consisting of an amino acid sequence having addition, deletion or substitution of one or several amino acids in the amino acid sequence represented by SEQ ID NO: 16 (particularly preferably, a polypeptide consisting of an amino acid sequence having substitution, deletion and/or addition, preferably deletion and/or addition of one or several amino acids in total at either one or both of the N terminal and the C terminal of the amino acid sequence represented by SEQ ID NO: 16); and a polypeptide consisting of an amino acid sequence having an amino acid identity of 80% or more, preferably 85% or more, more preferably 90% or more, 95% or more, 97% or more, 98% or more or 99% or more with the amino acid sequence represented by SEQ ID NO: 16. Further examples of said other polypeptides that can be used herein include a PAP-active fragment of at least one polypeptide selected from the group consisting of the polypeptide consisting of the amino acid sequence represented by SEQ ID NO: 16; a polypeptide consisting of an amino acid sequence having addition, deletion or substitution of one or several amino acids in the amino acid sequence represented by SEQ ID NO: 16; and a polypeptide consisting of the amino acid sequence having the aforementioned amino acid identity with the amino acid sequence represented by SEQ ID NO: 16. The fragment may be a polypeptide consisting of amino acids of preferably 250 or more, more preferably 300 or more, more preferably 400 or more and more preferably 450 or more. The “amino acid identity” refers to the percentage (%) of the number of identical amino acid residues relative to the total number of amino acid residues of the protein represented by SEQ ID NO: 16 when two amino acid sequences are aligned and arranged so as to obtain a highest degree of matching of amino acids between the two sequences, if necessary by introducing a gap. Furthermore, the substitution of amino acids is made preferably in accordance with conservative amino acid substitution. The preferable range of “several”, method for calculating amino acid identity and conservative amino acid substitution mentioned herein are the same as described with respect to GSH I. The aforementioned polypeptides each may be appropriately chemically modified.

The nucleotide sequence of a gene (DNA or RNA) encoding the PAP that can be used for preparation of PAP is not limited to the nucleotide sequence represented by SEQ ID NO: 15. The nucleotide sequence may be any nucleotide sequence encoding the amino acid sequence of a desired PAP and being suited to the host organism.

### <Preparation of Enzymes>

The methods for obtaining the aforementioned individual enzymes used in the present invention are not limited. Each of the enzymes can be prepared from organisms having the activity of the enzyme, for example, a wild strain or variant strain of a microorganism. As the organism having the activity of a desired enzyme, either an organism that natively has the enzymatic activity or an organism that has been augmented in the enzymatic activity may be used. Examples of the organism that has been augmented in the enzymatic activity include recombinant biological cells, in which the expression of a gene encoding each of the enzymes has been augmented by genetic engineering technique. Note that “the organism that has been augmented in the enzymatic activity” include not only an organism that natively has the enzymatic activity, in which the enzymatic activity has been augmented further, but also an organism that natively lacks in the enzymatic activity, in which the enzymatic activity has been added.

The recombinant biological cell obtained by genetic engineering technique typically refers to a recombinant biological cell having an ability to produce a desired enzyme. Such a cell can be obtained by inserting a gene (DNA or RNA) encoding the desired enzyme into an appropriate vector to prepare a recombinant vector and transforming an appropriate host cell with the recombinant vector. The desired enzyme can be produced by culturing the recombinant biological cell. Examples of the host cell include bacterial cells, yeast cells, filamentous fungus cells, plant cells and animal cells. In view of introduction and expression efficiency, bacterial cells are preferable, and particularly Escherichia coli cell is preferable.

### <Step A and Step A′>

A method for producing oxidized γ-glutamylcysteine according to the present invention is characterized by comprising step A of producing oxidized γ-glutamylcysteine by reacting L-cystine and L-glutamic acid in the presence of at least one enzyme selected from the group consisting of γ-glutamylcysteine synthetase and bifunctional glutathione synthetase, and ATP.

The method for producing oxidized γ-glutamylcysteine according to the present invention is also characterized by comprising step A′ of producing oxidized γ-glutamylcysteine by reacting L-cystine and L-glutamic acid. Step A′ may be a step based on an enzymatic reaction or a step based on a chemical reaction using no enzyme; preferably a step based on an enzymatic reaction and particularly preferably the step A as mentioned above. The enzymatic reaction is advantageous over the chemical synthesis reaction, for the reason that e.g., protection of a substrate compound with a functional group is not required, or specificity of a reaction is high.

Examples of step A′ based on a chemical reaction using no enzyme include, but not particularly limited to, a step comprising reacting L-cystine having two carboxyl groups protected with an appropriate protecting group with L-glutamic acid having an α-carboxyl group and an amino group protected with appropriate protecting groups and subjecting each of the two amino groups of one L-cystine molecule and a γ-carboxyl group of one L-glutamic acid molecule to dehydration condensation to form peptide bonds. In the step, if necessary, one or more protecting groups are removed after the dehydration condensation reaction. The protecting group for a carboxyl group may be a commonly known protecting group for a carboxyl group, such as a benzyl group. The protecting group for an amino group may be a commonly known protecting group for an amino group, such as t-butoxycarbonyl (Boc) group and a 9-fluorenylmethoxycarbonyl (Fmoc) group.

Step A may use, as GSH I and/or GSH F, a cell of an organism having GSH I and/or GSH F activity, which may be a living cell or may be a dead but undamaged cell. Alternatively, the step may use GSH I and/or GSH F present outside the cell, more specifically, a ground material (i.e., crushed material) of the cell of the organism. Alternatively, the step may use GSH I and/or GSH F protein, which is isolated from the cell and purified. Herein, the purification degree of a protein having GSH I and/or GSH F activity is not limited, and the step may use GSH I and/or GSH F roughly purified. Step A preferably uses no living cell having GSH I and/or GSH F activity, and more preferably, uses neither a living cell nor an undamaged dead cell having GSH I and/or GSH F activity. In particular, step A preferably uses the extracellular GSH I and/or GSH F, more specifically, GSH I and/or GSH F in a cell ground material or GSH I and/or GSH F protein which has been isolated from a cell and purified, because in this case, in comparison with the case of using a living cell, it is considered that starting L-cystine and the reaction product, oxidized γ-glutamylcysteine, are easily maintained in an oxidized state in the reaction mixture and thus oxidized γ-glutamylcysteine can be efficiently obtained. In the case where step A uses extracellular GSH I and/or GSH F, in which a reduction action due to a living cell is not present in the reaction system, the above action presumably takes place. This tendency is particularly remarkable when the reaction mixture is placed in the conditions where the reaction mixture is allowed to be in contact with air. Furthermore, in the case where step A uses a living cell having GSH I and/or GSH F activity, adenosine monophosphate (AMP) tends to be easily decomposed in the reaction system. If AMP, which is one of the intermediates of the ATP regeneration reaction (described later), is decomposed, the efficiency of ATP regeneration reaction would decrease. In contrast, in the case where step A uses extracellular GSH I and/or GSH F, AMP is rarely decomposed and the ATP regeneration reaction can efficiently proceed. Because of this, use of extracellular GSH I and/or GSH F is preferable. More specifically, when step A of the present invention is carried out by using extracellular GSH I and/or GSH F, decomposition of AMP and reduction of oxidized γ-glutamylcysteine can be simultaneously suppressed.

In the specification, “ground” (i.e., “crushed”) of a cell refers to a treatment by which the surface structure of a cell is damaged to the extent that an enzyme produced within the cell is accessible from outside the cell, and the cell is not necessarily fragmented. In the specification, “ground material” (“crushed material”) of a cell refers to a ground cell resulting from the grinding treatment. Grinding (crushing) treatment of a cell can be carried out by applying a single grinding treatment or a plurality of grinding treatments in an appropriate order. Examples of the grinding treatment of a cell include a physical treatment, a chemical treatment and an enzymatic treatment. Examples of the physical treatment include treatments using a high-pressure homogenizer, an ultrasonic homogenizer, a French press, a ball mill and a combination of these treatments. Examples of the chemical treatment include a treatment using an acid (preferably a strong acid), such as hydrochloric acid and sulfuric acid, a treatment using a base (preferably a strong base), such as sodium hydroxide and potassium hydroxide, and a combination of these treatments. Examples of the enzymatic treatment include treatments using, lysozyme, zymolyase, glucanase, protease and cellulase, and a combination of these treatments.

L-cystine and L-glutamic acid used as a substrate in step A and/or step A′ and ATP used in step A each can be added in the reaction system in various forms, such as a salt, free form and a solvate (e.g., a hydrate).

The reaction system (for example, reaction mixture) in step A and/or step A′ does not substantially contain L-cysteine. More specifically, in the reaction system of step A and/or step A′, L-cystine is contained in a percentage of 70 mol % or more relative to the total molar amount of L-cystine and L-cysteine, more preferably 80 mol % or more, more preferably 90 mol % or more, further preferably 95 mol % or more, further preferably 98 mol % or more and most preferably 100 mol %. The requirement that the percentage of L-cystine relative to the total molar amount of L-cystine and L-cysteine falls within the above range is satisfied preferably at least at the starting time of the reaction in step A and/or step A′ and more preferably during the period from the starting time to completion of the reaction in step A and/or step A′.

In step A, GSH I and/or GSH F is reacted with a reaction system containing L-cystine and L-glutamic acid in the presence of ATP. The reaction can be carried out in a reaction mixture containing a solvent, such as water, controlled at appropriate pH. The conditions at this time are not limited; however a substrate concentration (total concentration of L-cystine and L-glutamic acid) can be set to be preferably about 0.1 to 99 wt % and more preferably 1 to 20 wt %. At the starting time of the reaction, the quantitative ratio (molar ratio) of L-cystine and L-glutamic acid in the substrate can be L-cystine:L-glutamic acid=1:about 2, for example, L-cystine:L-glutamic acid=1:1 to 4, and preferably 1:1.5 to 3. The reaction temperature can be preferably 10 to 60° C. and more preferably 20 to 50° C. The pH of the reaction is preferably 4 to 11 and more preferably 6 to 9. The reaction time can be preferably 1 to 120 hours and more preferably 1 to 72 hours. The reaction in step A and/or step A′ is preferably carried out while allowing the reaction mixture in contact with air, because, under this condition, starting L-cystine and the reaction product, oxidized γ-glutamylcysteine, are easily maintained in an oxidized state. After completion of the reaction in step A and/or step A′, reduced γ-glutamylcysteine is not substantially contained in the reaction system (for example, reaction mixture), more specifically, oxidized γ-glutamylcysteine is contained in a percentage of 70 mol % or more relative to the total molar amount of oxidized γ-glutamylcysteine and reduced γ-glutamylcysteine in the reaction system after completion of the reaction in step A and/or step A′, more preferably 80 mol % or more, more preferably 90 mol % or more, further preferably 95 mol % or more, further preferably 98 mol % or more, and most preferably 100 mol %.

The concentration of each of the enzymes in the reaction mixture can be appropriately controlled. For example, the lower limit thereof in terms of protein is 1 μg/ml or more and the upper limit is not specified; however, it can be appropriately controlled in the range up to preferably 100 mg/ml or less. When GSH I is used in step A, the GSH I activity in the reaction mixture in step A is not limited; however, the lower limit thereof is preferably 0.1 U/ml or more and the upper limit (not particularly specified) can be usually 10000 U/ml or less. When GSH F is used in step A, GSH F activity in the reaction mixture in step A is not limited; however, the lower limit thereof is preferably 0.1 U/ml or more and the upper limit (not particularly specified) can be set at usually 10000 U/ml or less.

The concentration of ATP in the reaction mixture can be appropriately controlled depending upon the concentration of cystine serving as a substrate and the presence or absence of the ATP regenerating system. In step A, ATP is consumed twice by molar ratio compared to cystine. When step A is carried out in conjugation with the ATP regeneration reaction, the addition amount of ATP relative to cystine can be greatly reduced. Then, the upper limit of the ATP concentration in the reaction mixture in step A, which is not limited, is preferably 4 fold or less and more preferably 2.2 fold or less as large as the cystine molar concentration. The lower limit of the ATP concentration in the reaction mixture in step A, which is not limited, is preferably 0.0001 fold or more, more preferably 0.001 fold or more and further preferably 0.01 fold or more as large as the cystine molar concentration.

### <Step B and Step B′>

A method for producing oxidized glutathione (GSSG) according to the present invention is characterized by comprising step B of reacting oxidized γ-glutamylcysteine and glycine in the presence of glutathione synthetase (GSH II) and ATP to produce oxidized glutathione.

A method for producing GSSG according to the present invention is also characterized by comprising step B′ of reacting oxidized γ-glutamylcysteine and glycine in the presence of GSH II and ATP to produce oxidized glutathione. Step B′ may be a step based on an enzymatic reaction or a step based on a chemical reaction using no enzyme. Step B′ is preferably a step based on an enzymatic reaction and is particularly preferably the step B mentioned above. The enzymatic reaction is advantageous over the chemical synthesis reaction, for the reason that e.g., protection of a substrate compound with a functional group is not required, or specificity of a reaction is high.

Examples of step B′ based on a chemical reaction using no enzyme include, but not particularly limited to, a step comprising reacting oxidized γ-glutamylcysteine, in which an α-carboxyl group and an amino group in an L-glutamic acid residue are protected with appropriate protecting groups, and glycine, in which a carboxyl group is protected with an appropriate protecting group, and subjecting each of the two carboxyl groups in one oxidized γ-glutamylcysteine molecule and an amino group of one glycine molecule to dehydration condensation to form a peptide bond. In the step, if necessary, one or more protecting groups are removed after the dehydration condensation reaction. The protecting group for a carboxyl group may be a commonly known protecting group for a carboxyl group, such as a benzyl group. The protecting group for an amino group may be a commonly known protecting group for an amino group, such as t-butoxycarbonyl (Boc) group and a 9-fluorenylmethoxycarbonyl (Fmoc) group.

Step B may use, as GSH II, a cell of an organism having GSH II activity, which may be a living cell or may be a dead but undamaged cell. Alternatively, the step may use GSH II present outside the cell, more specifically, a ground material (i.e., crushed material) of the cell of the organism. Alternatively, the step may use GSH II protein, which is isolated from the cell and purified. Herein, the purification degree of a protein having GSH II activity is not limited, and the step may use GSH II roughly purified. Herein, the “ground material” of a cell is the same as defined above. Step B preferably uses no living cell having GSH II activity, and more preferably, uses neither a living cell nor an undamaged dead cell having GSH II activity. In particular, step B preferably uses the extracellular GSH II, more specifically, GSH II in a cell ground material or GSH II protein which has been isolated from a cell and purified, because in this case in comparison with the case of using a living cell, it is considered that starting oxidized γ-glutamylcysteine and the reaction product, oxidized glutathione, are easily maintained in an oxidized state in the reaction mixture and thus oxidized glutathione can be efficiently obtained. In the case where step B uses the extracellular GSH II, in which a reduction action due to a living cell is not present in the reaction system, the above action presumably takes place. This tendency is particularly remarkable when the reaction mixture is placed in the conditions where the reaction mixture is allowed to be in contact with air. Furthermore, in the case where step B uses a living cell having GSH II activity, adenosine monophosphate (AMP) tends to be easily decomposed in the reaction system. If AMP is decomposed, the efficiency of ATP regeneration reaction would decrease. In contrast, in the case where step B uses extracellular GSH II, AMP is rarely decomposed and the ATP regeneration reaction can efficiently proceed. Because of this, use of extracellular GSH II is preferable. More specifically, when step B of the present invention is carried out by using extracellular GSH II, decomposition of AMP and reduction of oxidized glutathione and oxidized γ-glutamylcysteine can be simultaneously suppressed.

Oxidized γ-glutamylcysteine and glycine used as substrates in Step B and/or step B′ and ATP used in step B can be added in a reaction system in various forms, such as a salt, free form and a solvate (e.g., a hydrate).

The reaction system (for example, the reaction mixture) in step B and/or step B′ does not substantially contain reduced γ-glutamylcysteine. More specifically, in the reaction system of step B and/or step B′, oxidized γ-glutamylcysteine is contained in a percentage of 70 mol % or more relative to the total molar amount of oxidized γ-glutamylcysteine and reduced γ-glutamylcysteine, more preferably 80 mol % or more, more preferably 90 mol % or more, further preferably 95 mol % or more, further preferably 98 mol % or more and most preferably 100 mol %. The requirement that the percentage of oxidized γ-glutamylcysteine relative to the total molar amount of oxidized γ-glutamylcysteine and reduced γ-glutamylcysteine falls within the above range is satisfied preferably at least at the starting time of the reaction in step B and/or step B′ and more preferably during the period from the starting time to completion of the reaction in step B and/or step B′.

In step B, GSH II is reacted with a reaction system containing oxidized γ-glutamylcysteine and glycine in the presence of ATP. The reaction can be carried out in a reaction mixture containing a solvent, such as water, controlled at appropriate pH. The conditions at this time are not limited; however, a substrate concentration (total concentration of γ-glutamylcysteine and glycine) can be set to be preferably about 0.1 to 99 wt % and more preferably 1 to 20 wt %. At the starting time of the reaction, the quantitative ratio (molar ratio) of oxidized γ-glutamylcysteine and glycine in the substrate can be oxidized γ-glutamylcysteine:glycine=1:about 2, for example, oxidized γ-glutamylcysteine:glycine=1:1 to 4, and preferably 1:1.5 to 3. The reaction temperature can be preferably 10 to 60° C. and more preferably 20 to 50° C. The pH of the reaction is preferably 4 to 11 and more preferably 6 to 9. The reaction time can be preferably 1 to 120 hours and more preferably 1 to 72 hours. The reaction in step B and/or step B′ is preferably carried out while allowing the reaction mixture in contact with air, because, under this condition, starting oxidized γ-glutamylcysteine and the reaction product, oxidized glutathione, are easily maintained in an oxidized state. After completion of the reaction in step B and/or step B′, reduced glutathione is not substantially contained in the reaction system (for example, the reaction mixture), more specifically, oxidized glutathione is contained in a percentage of 70 mol % or more relative to the total molar amount of oxidized glutathione and reduced glutathione, in the reaction system after completion of the reaction in step B and/or step B′, more preferably 80 mol % or more, more preferably 90 mol % or more, further preferably 95 mol % or more, further preferably 98 mol % or more, and most preferably 100 mol %.

Oxidized γ-glutamylcysteine serving as a starting material in step B can be obtained in step A. Oxidized γ-glutamylcysteine may be separated from the reaction mixture after completion of step A and then used in step B. After completion of step A, step B may be carried out without separating oxidized γ-glutamylcysteine by adding GSH II and glycine to the reaction mixture of the step A. Alternatively, step A is carried out in the conditions deficient in at least one of GSH II and glycine, in other words, in the conditions in which step B cannot proceed; and then step B may be started by supplementing the deficient element in the above reaction, to the reaction mixture without separating oxidized γ-glutamylcysteine from the reaction mixture after completion of step A.

The reactions of step A and step B are not necessarily carried out in order and may be simultaneously carried out. More specifically, a starting mixture containing L-cystine, L-glutamic acid and glycine may be reacted in the presence of the enzymes used in step A and the enzyme used in step B and ATP. This embodiment is also one of the embodiments of the present invention in which oxidized γ-glutamylcysteine used as a starting material in step B, is produced by step A.

Similarly, oxidized γ-glutamylcysteine serving as a starting material in step B′ can be obtained in step A′. Oxidized γ-glutamylcysteine may be separated from the reaction mixture after completion of step A′ and then used in step B′. After completion of step A′, step B′ may be carried out without separating oxidized γ-glutamylcysteine by adding glycine to the reaction mixture of step A′ and appropriately controlling reaction conditions. The reactions of step A′ and step B′ are not necessarily carried out in order and may be simultaneously carried out. More specifically, a starting mixture containing L-cystine, L-glutamic acid and glycine may be reacted. This embodiment is also one of the embodiments of the present invention in which oxidized γ-glutamylcysteine serving as a starting material in step B′ is produced by step A′.

The concentration of each of the enzymes in a reaction mixture can be appropriately controlled. For example, the lower limit thereof in terms of protein is 1 μg/ml or more and the upper limit is not particularly specified; however, it can be appropriately controlled in the range up to preferably 100 mg/ml or less. When step B uses GSH II, the GSH II activity in the reaction mixture in step B is not limited; however, the lower limit thereof is preferably 0.1 U/ml or more and the upper limit (not particularly specified) can be set at usually 10000 U/ml or less.

The concentration of ATP in the reaction mixture can be appropriately controlled depending upon the concentration of oxidized γ-glutamylcysteine serving as a substrate and the presence or absence of the ATP regenerating system. In step B, ATP is consumed twice by molar ratio compared to oxidized γ-glutamylcysteine. When step B is carried out in conjugation with the ATP regeneration reaction, the addition amount of ATP relative to oxidized γ-glutamylcysteine can be greatly reduced. Then, the upper limit of the ATP concentration in the reaction mixture in step B, which is not limited, is preferably 4 fold or less and more preferably 2.2 fold or less as large as the molar concentration of oxidized γ-glutamylcysteine. The lower limit of concentration of ATP in the reaction mixture in step B, which is not limited, is preferably 0.0001 fold or more, more preferably 0.001 fold or more and further preferably 0.01 fold or more as large as the molar concentration of oxidized γ-glutamylcysteine.

### <ATP Regeneration Reaction>

Steps A and B each are a step in which ATP is consumed to produce ADP. Since ATP is a relatively expensive material, steps A and B are preferably carried out in conjugation with an ATP regeneration reaction for regenerating ATP from ADP produced in the steps.

The ATP regeneration reaction includes a reaction for regenerating ATP from ADP using a phosphate group supply source and phosphotransferase.

A scheme of ATP regeneration reaction is shown below. The scheme shows an ATP regeneration reaction using a polyphosphoric acid (condensed phosphoric acid) as a phosphate group supply source and a combination of polyphosphate-dependent AMP transferase (PAP) and adenylate kinase (ADK) as a phosphotransferase.

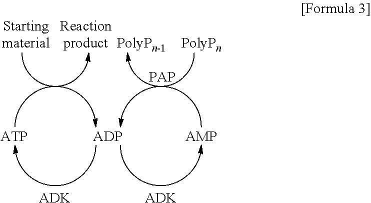

In the scheme, AMP represents adenosine monophosphate, PAP a polyphosphate-dependent AMP transferase, ADK an adenylate kinase, PolyPₙ a polyphosphoric acid (condensed phosphoric acid) in which “n” represents the number of phosphorus atoms, PolyPₙ₋₁ a polyphosphoric acid (condensed phosphoric acid) in which “n−1” represents the number of phosphorus atoms, “starting material” a starting material of step A or B and “reaction product” a reaction product in step A or B.

More specifically, when step A and B are carried out in the presence of a polyphosphoric acid, PAP and ADK, ATP is consumed to produce ADP; ADP is then converted into ATP and AMP by the action of ADK; and AMP produced by the action of ADK is converted into ADP by the action of PAP. This ATP regeneration reaction can be carried out in conjugation with the reactions of steps A and B.

This reaction may use, as ADK and PAP, a cell of an organism having the activities of these enzymes, which may be living or may be dead but undamaged. Alternatively, the reaction may use ADK and/or PAP present outside the cell, more specifically, a ground material (i.e., crushed material) of the cell. Alternatively, the reaction may use ADK and/or PAP protein, which is isolated from the cell and purified. Herein, the degree of purification of a protein having ADK or PAP activity is not limited, and the reaction may use ADK or PAP roughly purified. The “ground material” of a cell is the same as defined above. Step A or B preferably uses no living cell having ADK and/or PAP activity, and more preferably, uses neither a living cell nor an undamaged dead cell having ADK and/or PAP activity.

The ATP regeneration reaction preferably uses, as the enzyme, extracellular ADK and/or PAP, more specifically, ADK and/or PAP present in a cell ground material or ADK and/or PAP protein isolated from the cell. When the ATP regeneration reaction uses a living cell having ADK and/or PAP activity, AMP tends to be easily decomposed. When AMP is decomposed, the efficiency of ATP regeneration reaction would decrease. In contrast, in the case where the ATP regeneration reaction uses extracellular ADK and/or PAP, AMP is rarely decomposed and the ATP regeneration reaction can efficiently proceed. Because of this, use of extracellular ADK and/or PAP is preferable. Note that, the case where step A and/or step B is carried out without using a living cell is advantageous since a reduction action due to a living cell is not present and thus reduction of oxidized γ-glutamylcysteine and/or oxidized glutathione is suppressed. More specifically, when step A and/or step B of the present invention is carried out while using extracellular ADK and/or PAP in the ATP regeneration reaction, decomposition of AMP and reduction of oxidized γ-glutamylcysteine and/or oxidized glutathione can be simultaneously suppressed.

The concentration of each of the enzymes in the reaction mixture used in the ATP regeneration reaction can be appropriately controlled. For example, the lower limit thereof in terms of protein is 1 μg/ml or more and the upper limit is not particularly specified; however, it can be appropriately controlled in the range up to preferably 100 mg/m or less. When step A or B is carried out in conjugation with the ATP regeneration reaction, the ADK activity in the reaction mixture is not limited; however, the lower limit thereof is preferably 5 U/ml or more. The upper limit is not particularly specified; however, it can be usually set to be 500000 U/ml or less. The PAP activity in the reaction mixture is not limited; however, the lower limit thereof is preferably 1 U/ml or more. The upper limit is not particularly specified, however, the upper limit can be usually set to be 100000 U/ml or less.

The addition amount of polyphosphoric acid (condensed phosphoric acid) may be appropriately controlled in accordance with the amount of substrate in the reaction. The polyphosphoric acid can be added in various forms, such as a salt (e.g., sodium salt, potassium salt), free form and a solvate (e.g., a hydrate). The degree of polymerization (the number of phosphorus atoms per molecule) of the polyphosphoric acid is not limited. Sodium metaphosphate used in Examples and Comparative Examples was a mixture of various condensed sodium phosphates different in degree of polymerization.

### EXAMPLES

### Experiment 1

Preparation of γ-Glutamylcysteine Synthetase (GSH I) Derived from Escherichia coli K12 Strain

A DNA primer (Primer-1: SEQ ID NO: 2) having a restriction enzyme SacI cleavage site and an SD sequence bound to the nucleotide sequence corresponding to the N terminal portion of the GSH I gene (SEQ ID NO: 1); and a DNA primer (Primer-2: SEQ ID NO: 3) having a restriction enzyme KpnI cleavage site bound to the nucleotide sequence corresponding to the C terminal portion of the GSH I gene (SEQ ID NO: 1) were prepared. Using these DNA primers, DNA between these sequences was amplified by PCR to obtain a DNA fragment containing a full length of the GSH I gene. The template used in the PCR amplification was the genomic DNA of Escherichia coli K12 strain. The result of analysis of the nucleotide sequence of the obtained DNA fragment showed that the full length of GSH I gene (SEQ ID NO: 1) was contained. The obtained DNA fragment was inserted between a SacI recognition site and a KpnI recognition site present at the downstream of a lac promoter of plasmid pUC18 (GenBank Accession No. L09136, manufactured by Takara Bio Inc.) to construct a recombinant vector, pUCGSHI. E. coli HB101 competent cell (manufactured by Takara Bio Inc.) was transformed with the recombinant vector pUCGSHI to obtain E. coli HB101 (pUCGSHI). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH I activity was 5 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### Experiment 2

Preparation of Glutathione Synthetase (GSH II) Derived from Escherichia coli K12 Strain

A DNA primer (Primer-3: SEQ ID NO: 5) having a restriction enzyme NdeI cleavage site bound to the nucleotide sequence corresponding to the N terminal portion of a GSH II gene (SEQ ID NO: 4); and a DNA primer (Primer-4: SEQ ID NO: 6) having a restriction enzyme EcoRI cleavage site bound to the nucleotide sequence corresponding to the C terminal portion of GSH II gene (SEQ ID NO: 4) were prepared. Using these DNA primers, DNA between these sequences was amplified by PCR to obtain a DNA fragment containing a full length of the GSH II gene. The template used in the PCR amplification was the genomic DNA of Escherichia coli K12 strain. The result of analysis of the nucleotide sequence of the obtained DNA fragment showed that the full length of GSH II gene (SEQ ID NO: 4) was contained. The obtained DNA fragment was inserted between a NdeI recognition site and an EcoRI recognition site present at the downstream of a lac promoter of plasmid pUCN18 to construct a recombinant vector, pNGSHII. The plasmid pUCN18 was a plasmid obtained by substituting “T” at the 185th positions of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNGSHII to obtain E. coli HB101 (pNGSHII). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH II activity was 5 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### Experiment 3

Preparation of Bifunctional Glutathione Synthetase (GSH F) Derived from Streptococcus agalactiae

A GSH F gene fragment (SEQ ID NO: 7) derived from Streptococcus agalactiae was manufactured in accordance with a gene synthesis method (by Eurogentec). In this GSH F gene fragment, the codons were optimized for expression in Escherichia coli; and a restriction enzyme NdeI cleavage site was bound to the nucleotide sequence corresponding to the N terminal portion; and a restriction enzyme EcoRI cleavage site was bound to the nucleotide sequence corresponding to the C terminal portion. The obtained DNA fragment was inserted between a NdeI recognition site and an EcoRI recognition site present at the downstream of a lac promoter of plasmid pUCN18 to construct a recombinant vector, pNGSHF. The plasmid pUCN18 was a plasmid obtained by substituting “T” at the 185th position of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNGSHF to obtain E. coli HB101 (pNGSHF). The obtained transformant was inoculated on 50 ml of 2×YT culture medium (tryptone 1.6%, yeast extract 1.0%, NaCl 0.5%, pH7.0) containing 200 μg/ml ampicillin and subjected to shaking culture at 37° C. for 24 hours. Then, enzyme activity was measured. As a result, GSH F activity was 3 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### Experiment 4

Preparation of AMP Phosphotransferase (PAP) Derived from Acinetobacter johnsonii

A PAP gene fragment (SEQ ID NO: 8) was manufactured in accordance with a gene synthesis method (by Eurogentec). In this PAP gene fragment, the codons were optimized for expression in Escherichia coli; and a restriction enzyme NdeI cleavage site was bound to the nucleotide sequence corresponding to the N terminal portion; and a restriction enzyme EcoRI cleavage site was bound to the nucleotide sequence corresponding to the C terminal portion. The obtained DNA fragment was inserted between a NdeI recognition site and an EcoRI recognition site present at the downstream of a lac promoter of plasmid pUCN18 to construct a recombinant vector, pNPAP. The plasmid pUCN18 was a plasmid obtained by substituting “T” at the 185th position of pUC18 (GenBank Accession No. L09136 manufactured by Takara Bio Inc.) with “A” to destruct an NdeI site and further substituting “GC” at the 471-472nd positions with “TG” to newly introduce a NdeI site. E. coli HB101 competent cell (manufactured by Takara Bio Inc.) was transformed with the recombinant vector pNPAP to obtain E. coli HB101 (pNPAP). The obtained transformant was inoculated on 50 ml of 2×YT culture medium (tryptone 1.6%, yeast extract 1.0%, NaCl 0.5%, pH7.0) containing, 200 μg/ml ampicillin and subjected to shaking culture at 37° C. for 24 hours. Then, enzyme activity was measured. As a result, PAP activity was 40 U/ml and the activity of ADK derived from Escherichia coli used as a host cell was 90 U/ml. Subsequently, bacterial cells were centrifugally collected, suspended in 2.5 ml of a 100 mM phosphate buffer (pH7.0) and sonicated to obtain an enzyme solution.

### <Calculation of Yield>

A method of calculating the yield of each of the compounds obtained in the Experiments herein was as follows.

A reaction product was quantified based on analysis by high-performance liquid chromatography and then the yield thereof was obtained by the following expression:

Yield: the production amount (mol) of compound/starting L-cystine (mol)×100

The conditions of the above high-performance liquid chromatography are as follows. In the elution conditions, reduced glutathione (GSH), reduced γ-glutamylcysteine (γ-GC), oxidized γ-GC, oxidized glutathione (GSSG) sequentially elute in this order.

### [Analysis of Yield]

Column: ODS-HG-3 (4.6 mmφ×150 mm, manufactured by Nomura Chemical Co., Ltd.);

Eluent: Solution prepared by dissolving potassium dihydrogenphosphate (12.2 g) and sodium heptane sulfonate (3.6 g) in distilled water (1.8 L), controlling pH of the solution to be pH 2.8 with phosphoric acid and further adding methanol (186 ml);

Flow rate: 1.0 ml/minute;

Column temperature: 40° C.;

Measurement wavelength: 210 nm.

### Example 1

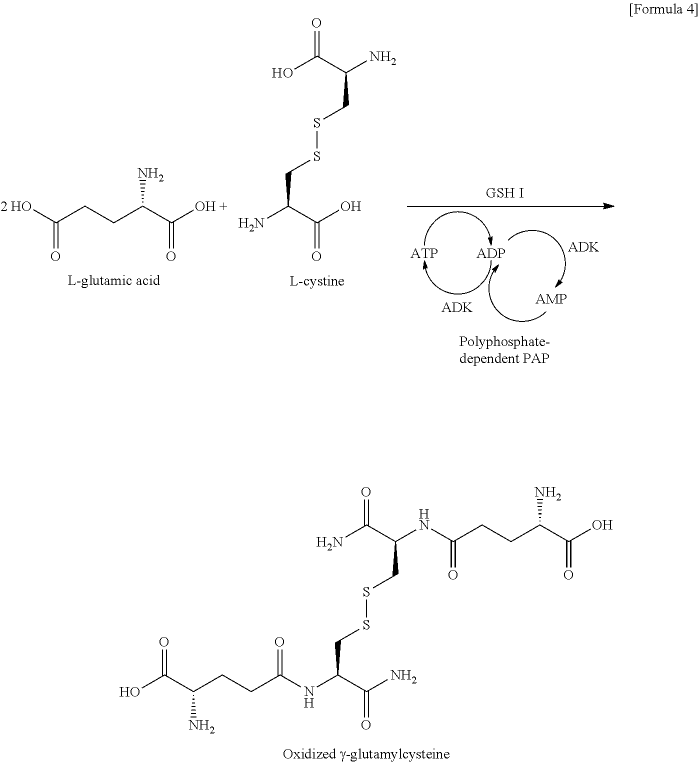

Monosodium L-glutamate monohydrate (0.3629 g (2.15 mmol)), L-cystine dihydrochloride (0.3113 g (0.99 mmol)), magnesium sulfate hepta hydrate (0.7079 g), ATP (0.0583 g (0.11 mmol)), sodium metaphosphate (0.8 g) and distilled water (12 g) were mixed. The pH of the mixture was controlled to be 7.5 with a 15 wt % aqueous sodium hydroxide solution (0.8 g). To the mixture, the γ-glutamylcysteine synthetase (GSH I) solution (2 g) prepared in Experiment 1 and the PAP solution (2 g) prepared in Experiment 4 were added and a reaction was initiated at a reaction temperature of 30° C. The reaction was carried out for one hour and then the reaction solution was analyzed. As a result, production of oxidized γ-glutamylcysteine was verified. The yield after a one-hour reaction based on the starting L-cystine was 20 mol % and the yield after a 22-hour reaction was 95 mol %. In this case, the yield of reduced γ-glutamylcysteine after the 22-hour reaction based on the starting L-cystine was 1 mol % or less.

Since the GSH I solution prepared in Experiment 1 and the PAP solution prepared in Experiment 4 contained ADK derived from Escherichia coli used as a host cell, it was not necessary to separately prepare ADK.

### Example 2

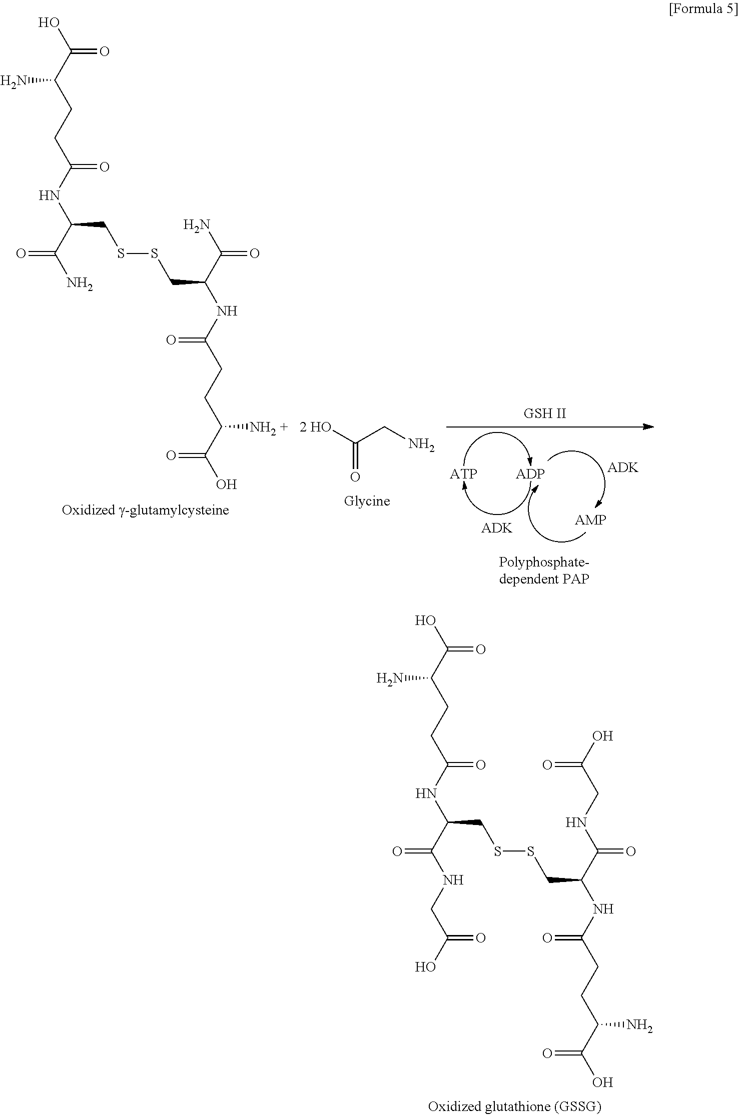

After the 22-hour reaction in Example 1, to the reaction solution, glycine (0.19 g (2.53 mmol)), the glutathione synthetase (GSH II) solution (2 g) prepared in Experiment 2 and the PAP solution (2 g) prepared in Experiment 4 were added and a reaction was initiated. In this case, the pH of the mixture was controlled to be 7.5 with a 15 wt % aqueous sodium hydroxide solution (0.2 g). The reaction was carried out for one hour and then the reaction solution was analyzed. As a result, production of oxidized glutathione was verified. The yield after a one-hour reaction based on the starting L-cystine was 20 mol % and the yield after a 6-hour reaction was 86 mol %. In this case, the yield of reduced glutathione after the 6-hour reaction based on the starting L-cystine was 1 mol % or less.

Since ADK derived from Escherichia coli used as a host cell was contained in the GSH II solution prepared in Experiment 2 and the PAP solution prepared in Experiment 4, it was not necessary to separately prepare ADK.

### Example 3

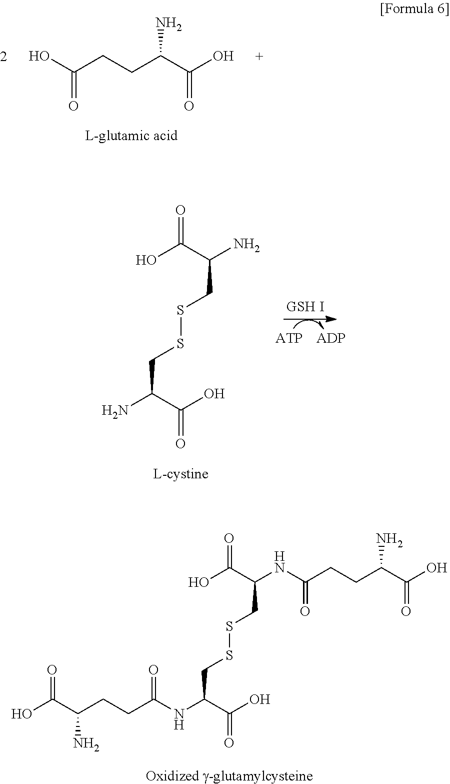 [SMILES: N[C@@H](CCC(=O)O)C(=O)O ; N[C@@H](CSSC[C@H](N)C(=O)O)C(=O)O ; N[C@@H](CCC(=O)N[C@@H](CSSC[C@H](NC(=O)CC[C@H](N)C(=O)O)C(=O)O)C(=O)O)C(=O)O]

Monosodium L-glutamate monohydrate (0.0717 g (0.42 mmol)), L-cystine dihydrochloride (0.0643 g (0.21 mmol)), magnesium sulfate hepta hydrate (0.2053 g), ATP (0.24 g (0.44 mmol)), and distilled water (17 g) were mixed. The pH of the mixture was controlled to be 7.5 with a 15 wt % aqueous sodium hydroxide solution (0.26 g). To the mixture, the γ-glutamylcysteine synthetase (GSH I) solution (2 g) prepared in Experiment 1 was added and a reaction was initiated at a reaction temperature of 30° C. The reaction was carried out for two hours and then the reaction solution was analyzed. As a result, production of oxidized γ-glutamylcysteine was verified. The yield after a two-hour reaction based on the starting L-cystine was 12 mol % and the yield after a 23-hour reaction was 95 mol %. In this case, the yield of reduced γ-glutamylcysteine after the 23-hour reaction based on the starting L-cystine was 1 mol % or less.

### Example 4

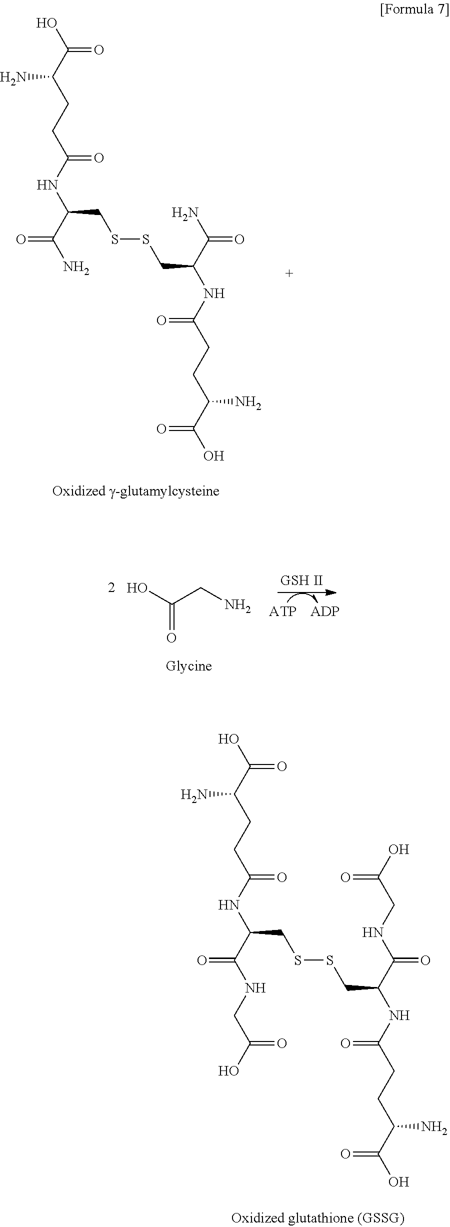 [SMILES: NC(=O)[C@H](CSSC[C@H](NC(=O)CC[C@H](N)C(=O)O)C(N)=O)NC(=O)CC[C@H](N)C(=O)O ; NCC(=O)O ; N[C@@H](CCC(=O)N[C@@H](CSSC[C@H](NC(=O)CC[C@H](N)C(=O)O)C(=O)NCC(=O)O)C(=O)NCC(=O)O)C(=O)O]

After the 23-hour reaction in Example 3, to the reaction solution, glycine (0.0426 g (0.57 mmol)), magnesium sulfate hepta hydrate (0.2064 g), ATP (0.2392 g (0.4 mmol)) and a glutathione synthetase (GSH II) solution (2 g) prepared in Experiment 2 were added and a reaction was initiated. In this case, the pH of the mixture was controlled to be 7.5 with a 15 wt % aqueous sodium hydroxide solution (0.2 g)). The reaction was carried out for one hour and then the reaction solution was analyzed. As a result, production of oxidized glutathione was verified. The yield after a one-hour reaction based on the starting L-cystine was 6 mol % and the yield after a 5-hour reaction was 70 mol %. In this case, the yield of reduced glutathione after the 5 hour reaction based on the starting L-cystine was 1 mol % or less.

### Example 5

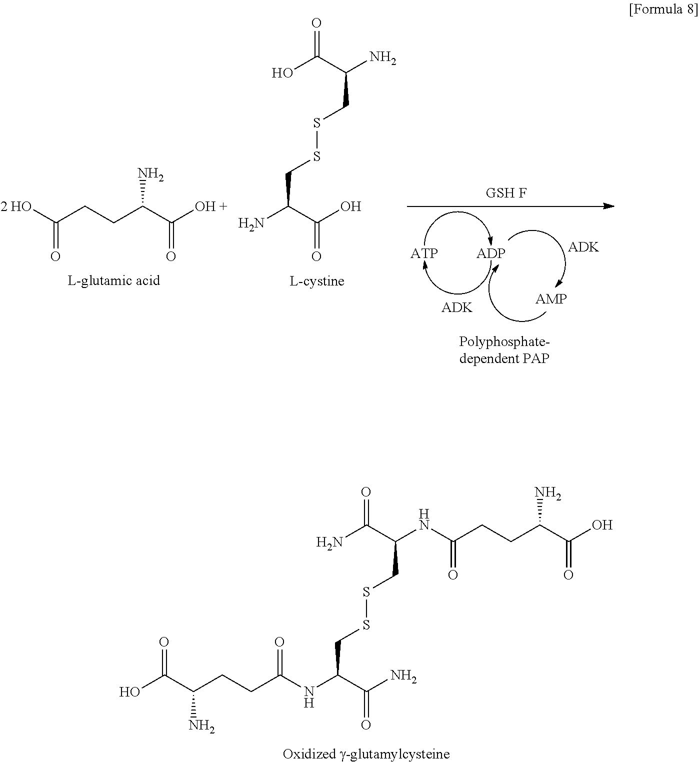

Monosodium L-glutamate monohydrate (0.3662 g (2.17 mmol)), L-cystine dihydrochloride (0.3121 g (1.00 mmol)), magnesium sulfate hepta hydrate (0.7019 g), ATP (0.058 g (0.11 mmol)), sodium metaphosphate (0.8 g) and distilled water (12 g) were mixed. The pH of the mixture was controlled to be 7.5 with a 15 wt % aqueous sodium hydroxide solution (0.8 g). To the mixture, the bifunctional glutathione synthetase (GSH F) solution (2 g) prepared in Experiment 3 and the PAP solution (2 g) prepared in Experiment 4 were added and a reaction was initiated at a reaction temperature of 30° C. The reaction was carried out for one hour and then the reaction solution was analyzed. As a result, production of oxidized γ-glutamylcysteine was verified. The yield after a one-hour reaction based on the starting L-cystine was 9 mol % and the yield after a 19-hour reaction was 61 mol %. In this case, the yield of reduced γ-glutamylcysteine after the 19-hour reaction based on the starting L-cystine was 1 mol % or less.

Since ADK derived from Escherichia coli used as a host cell was contained in the GSH F solution prepared in Experiment 3 and the PAP solution prepared in Experiment 4, it was not necessary to separately add ADK.

### SEQUENCE LISTING FREE TEXT

SEQ ID NO: 2: primer
SEQ ID NO: 3: primer
SEQ ID NO: 5: primer
SEQ ID NO: 6: primer

All publications including patents and patent applications cited in the specification are incorporated in the specification by reference in their entirety.

### SEQUENCE LISTING

1: A method for producing oxidized γ-glutamylcysteine, comprising:
reacting L-cystine and L-glutamic acid in the presence of adenosine triphosphate (ATP) and at least one enzyme selected from the group consisting of γ-glutamylcysteine synthetase and bifunctional glutathione synthetase, to produce oxidized γ-glutamylcysteine,
wherein a living cell does not perform reduction action in the reacting of L-cystine and L-glutamic acid.

2: The method according to claim 1, wherein the reacting of L-cystine and L-glutamic acid is carried out while a reaction mixture is in contact with air.

3: The method according to claim 1, wherein the reacting of L-cystine and L-glutamic acid is carried out in conjugation with an ATP regeneration reaction for regenerating adenosine diphosphate (ADP) into ATP.

4: The method according to claim 1, wherein the γ-glutamylcysteine synthetase is derived from Escherichia coli.

5: The method according to claim 1, wherein the bifunctional glutathione synthetase is derived from Streptococcus agalactiae.

6: A method for producing oxidized glutathione, comprising:
reacting oxidized γ-glutamylcysteine and glycine in the presence of glutathione synthetase and adenosine triphosphate (ATP) to produce oxidized glutathione,
wherein a living cell does not perform reduction action in the reacting of oxidized γ-glutamylcysteine and glycine.

7: The method according to claim 6, wherein the reacting of oxidized γ-glutamylcysteine and glycine is carried out while a reaction mixture is in contact with air.

8: The method according to claim 6, wherein the reacting of oxidized γ-glutamylcysteine and glycine is carried out in conjugation with an ATP regeneration reaction for regenerating adenosine diphosphate (ADP) into ATP.

9: The method according to claim 6, wherein the glutathione synthetase is derived from Escherichia coli.

10: The method according to claim 6, further comprising, prior to the reacting of oxidized γ-glutamylcysteine and glycine:
reacting L-cystine and L-glutamic acid to produce the oxidized γ-glutamylcysteine.

11: The method according to claim 10, wherein the reacting of L-cystine and L-glutamic acid is carried out in the presence of adenosine triphosphate (ATP) and at least one enzyme selected from the group consisting of γ-glutamylcysteine synthetase and bifunctional glutathione synthetase, wherein a living cell does not perform reduction action in the reacting of L-cystine and L-glutamic acid.

12: The method according to claim 11, wherein the reacting of L-cystine and L-glutamic acid is carried out in conjugation with an ATP regeneration reaction for regenerating adenosine diphosphate (ADP) into ATP.

13: The method according to claim 11, wherein the γ-glutamylcysteine synthetase is derived from Escherichia coli.

14: The method according to claim 11, wherein the bifunctional glutathione synthetase is derived from Streptococcus agalactiae.

15: The method according to claim 11, wherein the reacting of L-cystine and L-glutamic acid is carried out while a reaction mixture is in contact with air.

16: The method according to claim 1, wherein the γ-glutamylcysteine synthetase has an amino acid sequence having 95% or more identity to the amino acid sequence of SEQ ID NO: 9, and the bifunctional glutathione synthetase has an amino acid sequence having 95% or more identity to the amino acid sequence of SEQ ID NO: 12.

17: The method according to claim 6, wherein the glutathione synthetase has an amino acid sequence having 95% or more identity to the amino acid sequence of SEQ ID NO: 10.

18: The method according to claim 11, wherein the γ-glutamylcysteine synthetase has an amino acid sequence having 95% or more identity to the amino acid sequence of SEQ ID NO: 9, the bifunctional glutathione synthetase has an amino acid sequence having 95% or more identity to the amino acid sequence of SEQ ID NO: 12, and the glutathione synthetase has an amino acid sequence having 95% or more identity to the amino acid sequence of SEQ ID NO: 10.
